(19) United States
(12) Plant Patent Application Publication
(10) Pub. No.: US 2008/0184447 P1
(43) Pub. Date: Jul. 31, 2008

(54) HELIOTROPIUM PLANT NAMED 'HELIOVI'

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/652,791

(22) Filed: Jan. 12, 2007

(51) Int. Cl. A01H 5/00

(52) U.S. Cl. PLT/438

(57) ABSTRACT

A new Heliotropium plant particularly distinguished by its compact, good branching, and floriferous characteristics. The new variety can be used in 10-12 cm pots without use of growth regulators.

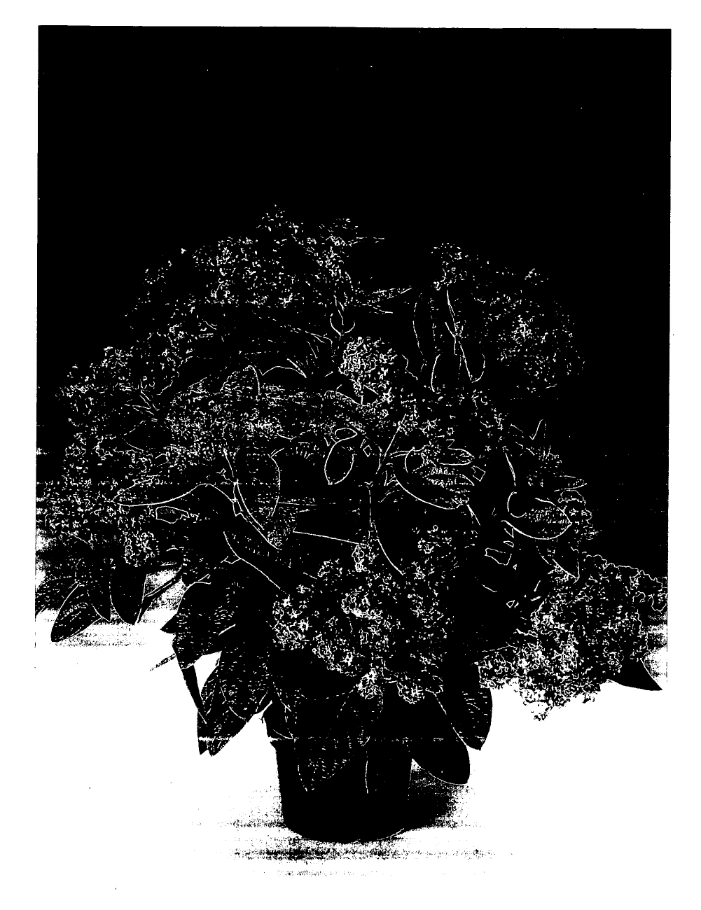

### LATIN NAME OF THE GENUS AND SPECIES OF THE PLANT CLAIMED

Heliotropium arborescens

### VARIETAL DENOMINATION

‘Heliovi’

### BACKGROUND OF THE NEW PLANT

The present invention relates to a new distinct variety of Heliotropium, botanically known as Heliotropium arborescens.

The new variety is propagated by cuttings resulting from a proprietary Heliotropium arborescens seedling identified as ‘B213-3,’ not patented. Seeds of ‘B213-3,’ resulting from open pollination, were harvested in 2001. The new variety ‘Heliovi’ was selected in 2002 as one of the plants of this population. Breeder's code of the new plant is ‘D3238-2.’

This new plant is an annual. The new variety is a product of a planned breeding program conducted in Enkhuizen, The Netherlands. The new cultivar ‘Heliovi’ is a compact, good branching variety that flowers throughout the summer. ‘Heliovi’ was asexually reproduced by cuttings for 4 years now and trialled under different conditions in Europe. It has retained its distinctiveness characteristics through successive propagation and its novelty is firmly fixed. The new variety reproduces true to type in successive generations of asexual reproduction.

### DESCRIPTION OF THE DRAWING

This new Heliotropium plant is illustrated by the accompanying photographic drawing which shows blooms, buds and foliage of the plant in full color, the color shown being as true as can be reasonably obtained by conventional photographic procedures.

### DESCRIPTION OF THE NEW VARIETY

The following detailed descriptions set forth the distinctive characteristics of this new Heliotropium plant. The data which defines these characteristics were collected from asexual reproductions carried out in Enkhuizen, Netherlands. The plant history was taken on 30 week old plants, grown in the open field in Enkhuizen, The Netherlands with day temperatures ranging from 16 and 30° Celsius, and night temperatures ranging from 10 to 16° Celsius.

Color references are primarily to the R.H.S. Color Chart of The Royal Horticultural Society of London.

TABLE 1
DIFFERENCES BETWEEN THE NEW VARIETY
‘HELIOVI’ AND TWO SIMILAR VARIETIES

| | ‘Atlanta’ | | |
|---|---|---|---|
| | (U.S. Plant | ‘Marine GWO’ | |
| | Patent No. 11,326) | (Not patented) | ‘Heliovi’ |
| Plant height | 25 cm | 60 cm | 28 cm |
| Plant width | 40-45 cm | 45 cm | 45-60 cm |
| Branching | Freely branching | Poor branching | Freely branching |
| Foliage color | RHS 145A | RHS 138A | Darker than RHS |
| | | | 148A |
| Inflorescence | 9-13 cm | 22 cm | Up to 16 cm |
| width | | | |
| Flower width | 8 mm | 7 mm | 9 mm |

### THE PLANT

Classification — botanical: Heliotropium arborescens
Parentage:
   Female parent.—Proprietary seedling of Heliotropium arborescens No. ‘B213-3,’ not patented.
   Male parent.—Unknown variety or seedling of Heliotropium arborescens.
Propagation:
   Type cuttings.—Terminal cuttings.
   Root description.—Fibrous.
   Rooting habit.—Freely branching.
   Time to initiate roots.—9-12 days at air temperature of 21° C.
   Time to develop roots.—14-18 days at air temperature of 17° C.
Plant description:
   Growth habit.—Upright.
   Plant.—Height: 28 cm Width: 45-60 cm.
   Strength.—Resistant to hot and cold weather.
   Branching character.—Freely branching without pinching.
   Number of branches per plant.—3-5 branches at base of plant +6-9 lateral branches.
   Crop time.—8 weeks.
The stem:
   Diameter.—Up to 8-9 mm.
   Shape.—Cylindrical.
   Length of.—Plant stem: Up to 40 cm Lateral branch: Up to 40 cm Internode: 4-17 cm depending on light conditions.
   Pubescence.—Densely, short hairs.
   Color.—Young stem: RHS 189A with anthocyan pigmentation RHS N199B Older stem: With anthocyan pigmentation RHS N1 99B.
The foliage:
   Arrangement.—Alternate.
   Number of leaves.—33.
   Shape of leaf.—Elliptic.
   Fragrance.—Light scent of hay.
   Leaf.—Apex: Acute Base: Acute Margin: Entire.
   Texture.—Densely covered on lower and upper side with soft, short hairs. Length: Up to 9 cm Width: 3 cm.
   Color.—Upper side Young leaf RHS 143A, darker than RHS 148A Old leaf RHS 177A Lower side: Young leaf RHS 197B, old leaf RHS 147C.
   Petiole.—Length: Up to 8 mm Color: RHS 197A Diameter: 1.5 mm Texture: Densely pubescent with short, soft hairs.
   Venation.—Shape Pinnate Color: RHS 187B — lower RHS 146D — upper.
Inflorescence:
   Flowering season.—Continuous and free flowering throughout spring summer, and fall.
   Type.—Terminal compound cymes.
   Height.—Up to 2 cm.
   Width.—1.5 cm when first flowers open; up to 16 cm when flowering is finished.
   Nr of open flowers per inflorescence.—Up to 100.
   Peduncle.—Strength: Strong Aspect: At an angle of 45° to the stem Length: 3-5 cm Diameter: 1-2 mm Texture: Densely, pubescent Color : RHS 200A.
   Fragrance.—Strong.
   Lastingness of inforescence on plant.—4 weeks.
Flower:
   Shape.—Funnel shaped.
   Length.—5mm.
   Width (of corolla).—9 mm.
   Nr of petals.—5, fused at base, forming tube.
   Apex.—Rounded.
   Margin.—Entire.
   Texture.—Upper side: Smooth Lower side: Moderate pubescence.
   Color.—Upper side: Young flower: RHS 83A, base: RHS 83B, old flower: darker than RHS N87A, base lighter than RHS N87D Lower side: Young flower: RHS 83C, old flower: RHS N87B.
   Corolla tube.—Length: 4 mm Diameter at base: 1 mm Diameter at tube opening: 1 mm Texture outer surface: Densely pubescent Texture inner surface: Smooth Color of outer surface: RHS 79A Color of inner surface: RHS 200A.
   Sepals.—Number: 5, fused at base Shape: Deltoid Length: 3 mm Width: 0.5 mm Apex: Acute to acuminate Margin: Entire Texture: Upper and lower surface densely pubescent with long soft hairs. Color: Darker than RHS N200A.
   Bud.—Shape: Tubular Length: 4mm Diameter: 1 mm Color: RHS 83D
Androecium:
   Anther.—Color: RHS N199D Length: 1 mm Shape: Ligulate
   Stamen.—Number: 5, fused to inside of tube Length: 0.3 mm.
   Pollen color.—RHS 155A.
   Amount of pollen.—Abundant.
Gynoecium:
   Pistil.—Number: One Length: 2.5 mm.
   Style.—Length: 1.0 mm Diameter: 0.2 mm Color: RHS 150C.
   Ovary.—Number : One per pistil Size: 0.8 mm diameter, 0.4 high Color: RHS 144B.
   Stigma.—Shape Capitate Color: RHS 144B, near top N144D.
Seed:
   Production.—Yes.
   Size.—Length: 1-1.5 mm Diameter: 0.7-1 mm.
   Color.—RHS 200A.
   Shape.—Ovoid.
Disease resistance and tolerance for weather conditions: Good tolerance to heat. No specific susceptibility for pests or diseases has been observed to date.

What is claimed is:

1. A new and distinct variety of Heliotropium plant substantially as illustrated and described herein.
